(19) United States
(12) Patent Application Publication
(10) Pub. No.: US 2026/0272988 A1
(43) Pub. Date: Sep. 17, 2026

(54) COMPOSITION COMPRISING MILK EXTRACELLULAR VESICLES AND GALACTOSE

(71) Applicant: FrieslandCampina Nederland B.V., Amersfoort (NL)

(72) Inventors: Anouk Leonie Feitsma, Wageningen (NL); Johannes Marie Wilhelmus Geurts, Deventer (NL); Elisabeth Gertruda Hendrika Maria Van Den Heuvel, Eindhoven (NL); Jacob Keijer, Ede (NL); Vincent Cornelis Johannis De Boer, Utrecht (NL)

(21) Appl. No.: 19/469,996

(22) PCT Filed: Mar. 28, 2024
(86) PCT No.: PCT/EP2024/058581

§ 371 Date: Sep. 26, 2025

(30) Foreign Application Priority Data
Mar. 29, 2023 (EP) 23165184.5

(51) Int. Cl. A61K 35/20; A61K 31/7004; A61K 45/06; C12N 15/113

(52) U.S. Cl. CPC A61K 35/20; A61K 31/7004; A61K 45/06; C12N 15/113; C12N 2310/141

(57) ABSTRACT

A synthetic nutritional composition comprising galactose and milk-derived extracellular vesicles (mEV) and uses thereof for increasing muscle growth.

### FIELD OF THE INVENTION

The invention relates to a synthetic nutritional composition comprising galactose and milk-derived extracellular vesicles (mEV), preferably bovine mEV, and the use thereof in enhancing muscle performance in a subject and/or in increasing muscle growth in a subject.

### BACKGROUND OF THE INVENTION

There are different energy sources in a muscle cell that generate ATP to contract muscle cells. Muscle cells initially use creatine phosphate, an energy-rich molecule stored in muscle tissue, to generate the energy-carrying molecule ATP. At moderate exercise level, mitochondria will be involved in ATP production but when exercise intensity is high (e.g. 40 meter sprint), creatine phosphate and glycolysis are used for energy production. In a few seconds after initiation of activity, the supplies of creatine phosphate will be depleted, forcing the bodies to break down glucose to provide ATP to contracting muscle cells for a few more minutes. If that is still not enough, then a slower, but more efficient way to generate ATP is relied upon. This slower way uses oxygen to burn fats and carbohydrates, in structures inside the cell called mitochondria that may be considered as an aerobic power plants.

Elite endurance athletes pack many more of these aerobic power plants into muscle cells than an average person. All elite athletes push themselves to the limit in search of small advantages, and for endurance athletes the mitochondria are a target for making marginal gains. Exercise not only promotes the generation of mitochondria, but also changes the structure and function of existing ones in ways that enhance physical stamina. Research shows that the mitochondria in leg muscles of endurance-trained athletes have more inner membrane folds (called cristae) than those of people who exercise recreationally, this increases the ratio of surface to mitochondrial volume. These cristae are where important enzymes attach and pass on electrons during cellular respiration; more folds means more oxygen uptake in muscle. A top endurance athlete should, therefore, boast more-efficient mitochondria than everyone else (A. King Nature Vol 592 1 Apr. 2021| S7-S9) Accordingly, there is a need to increase oxygen uptake of mitochondria, in particular for subjects in need of improved physical performance like athletes or subjects suffering from sarcopenia.

Sarcopenia is a type of muscle loss (muscle atrophy) that occurs with aging and/or immobility. It is characterized by the degenerative loss of skeletal muscle mass, quality, and strength. Loss of lean body mass such as skeleton muscle mass is also associated with increased risk of infection, decreased immunity, and poor wound healing. The weakness that accompanies muscle atrophy leads to higher risk of falls, fractures, physical disability, need for institutional care, reduced quality of life, is increased mortality, and increased healthcare costs. This represents a significant personal and societal burden and its public health impact is increasingly recognized. An increase of oxygen uptake by mitochondria may improve health and well being of people suffering from sarcopenia and hence contribute a sustainable development via an improved health and well-being.

Targeting mitochondria with specific dietary factors would be a convenient way to enhance muscle performance in subjects suffering from, or at risk of suffering from, various conditions, particularly sarcopenia and cardiac muscle injury. It would also be a convenient way to reduce chronic fatigue during or following the recovery from a viral infection. Therefore, it is desirable to develop nutritional intervention strategies to enhance muscle performance in subjects in need of improved physical performance, and more particularly in subjects suffering from or at risk of suffering from sarcopenia and/or chronic or acute cardiac damage, and/or to reduce chronic fatigue in subjects who are recovering or have recovered from a viral infection, such as COVID-19.

WO2022146743 discloses method of enhancing muscle performance in a subject in need of improved physical performance comprises administering an exosome-enriched product comprising intact bovine milk-derived exosomes to the subject in need thereof. It further discloses a method of reducing chronic fatigue in a subject recovering or recovered from a viral infection comprises administering an exosome-enriched product comprising intact bovine milk-derived exosomes to the subject. WO2022146743 also discloses a method for the preparation of exosome-enriched products e.g. in example 1.

The term “extracellular vesicle” or “EV” is herein defined as the generic term for lipid bilayer-delimited particles released from the cell and, unlike a cell, EVs cannot replicate. EVs range in diameter from near the size of the smallest physically possible unilamellar liposome (around 20-30 nanometers) to as large as 10 microns or more, although the vast is majority of EVs are smaller than 200 nm. They carry a cargo of proteins, nucleic acids, lipids, metabolites, and even organelles from the parent cell. Most cells that have been studied to date are thought to release EVs, including some bacterial, fungal, and plant cells that are surrounded by cell walls. A wide variety of EV subtypes have been proposed, defined variously by size, biogenesis pathway, cargo, cellular source, and function. Extracellular vesicles (EV or EVs) comprises exosomes (<100 nm) and microvesicles (100 nm-10 micron). EVs are present in biological fluids and are involved in multiple physiological and pathological processes. EVs are considered as an additional mechanism for intercellular communication allowing cells to exchange proteins, lipids and the genetic material. Milk derived extracellular vesicles (mEV) may be isolated using methods known in the art such as described by Blans et al (Blans et al Journal of Extracellular Vesicles, 2017 Vol. 6, 1294340 https://doi.org/ under 10.1080/20013078.2017.1294340 2017) or Tong et al (Tong et al Mol. Nutr. Food Res. 2020, 64, 1901251).

### SUMMARY OF THE INVENTION

It is therefore an object of the invention to provide a method which enhances muscle performance in a subject, in particular in a subject in need of improved physical performance. Another object of the invention is the provision of a method which increases muscle growth in a subject. It is another object of the invention to provide a method which reduces chronic fatigue in a subject who is recovering or has recovered from a viral infection.

The present invention is directed to a method of enhancing muscle performance in a subject in need of improved physical performance, comprising administering an extracellular vesicle enriched product comprising intact bovine milk-derived extracellular vesicles (mEV) to the subject in need thereof.

The present invention is also directed to a method of reducing chronic fatigue in a subject who is recovering or has recovered from a viral infection, comprising administering an extracellular vesicle enriched product comprising intact bovine mEV to the subject.

The invention further provides a composition that can be used in the methods of the invention.

The methods of the invention are advantageous in providing a convenient manner to improve mitochondrial function, and thereby improve muscle performance, in a subject in need of improved physical performance. The methods are useful in the prevention or treatment of conditions that are hallmarked by a reduction in spare respiratory capacity, including sarcopenia and chronic or acute cardiac damage.

The improved mitochondrial function afforded by the methods of the invention is also advantageous in that it reduces chronic fatigue during or following recovery from a viral illness associated with mitochondrial dysfunction, for example COVID-19. These and additional advantages of the inventive methods will be more fully apparent in view of the detailed description.

### BRIEF DESCRIPTION OF THE DRAWINGS

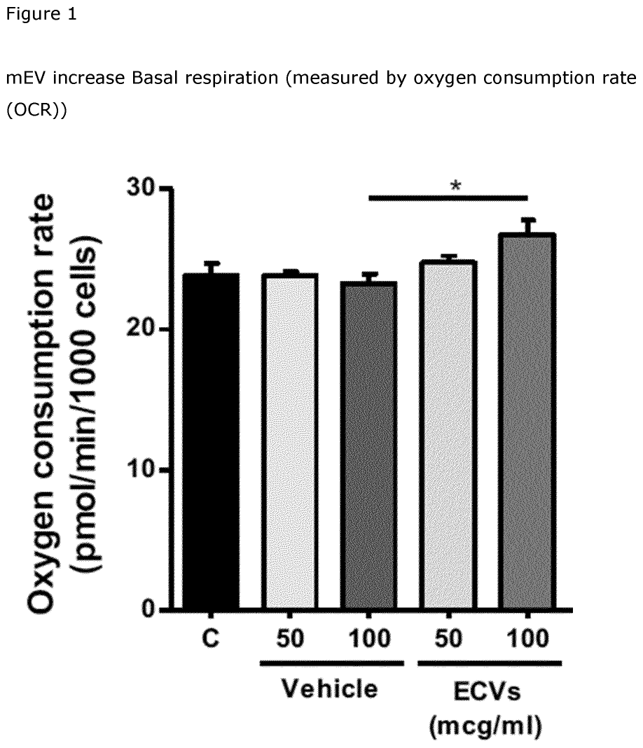

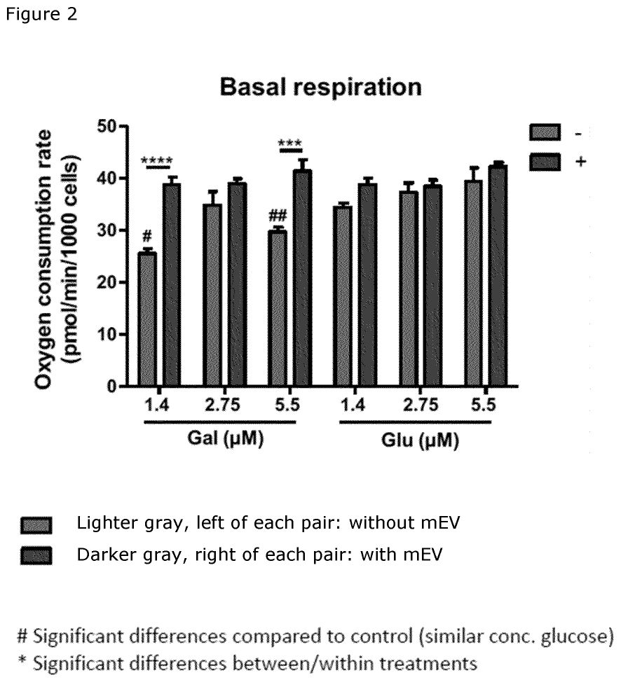

In FIG. 1 the basal respiration (OCR) as determined in a Seahorse experiment (example 2) is shown of sample 1 (second bar from the right) and sample 2 (right bar) as compared to control (C, left bar) and vehicle control (PBS; 5 and 10% of total volume; second and third bar from the left). The different samples are defined in Table 1 (example 1). Glucose and galactose each increase basal respiration as shown in FIG. 2 (galactose sample 6, 7, and 8 shown in 1^{st}, 3^{rd}, and 5^{th} bar, respectively, glucose sample 3, 4, and 5 shown in 7^{th}, 9^{th}, and 11^{th} bar, respectively.

### DETAILED DESCRIPTION

In a first aspect the invention relates to a synthetic nutritional composition comprising galactose and milk-derived extracellular vesicles (mEV). Milk-derived extracellular vesicles may be obtained using methods known in the art. By way of example, a gentle procedure of obtaining an mEV enriched product containing intact bovine milk-derived extracellular vesicles (mEV) may comprise physical methods and/or chemical methods.

In one embodiment, an mEV-enriched product is obtained by cascade membrane filtration. In a specific embodiment, the mEV-enriched product is lactose-free. In a specific embodiment, sweet cheese whey—which may be obtained by applying an enzyme or enzyme mixture, and more specifically a protease enzyme, for example chymosin, to milk to hydrolyze casein peptide bonds, thus allowing for enzymatic coagulation of casein in the milk paragraph—is processed using tandem multiple ceramic filtration steps. In a specific embodiment, a multiple filtration process employs, successively, membranes with cut offs which gradually decrease in size with each filtration step. In a specific embodiment, the method of processing sweet cheese whey is subjected to microfiltration (MF), ultrafiltration (UF) and diafiltration (DF). In one more specific embodiment, the process employs, successively, MF, UF and DF membranes with cut offs of about 1.4 μm, 0.14 μm and 10 kDa to provide an mEV enriched product.

The mEV-enriched product e.g. resulting from successive filtration steps, may be pasteurized to provide storage stability. For example, the mEV-enriched product may be heated, for example, at about 70° C. for about 15 seconds, to ensure microbiological stability in order to yield a pasteurized fraction. Other pasteurization conditions will be apparent to those skilled in the art and may be employed.

With or without pasteurization, the mEV-enriched product may be used as is or subjected to additional processing steps to provide a desired physical form.

As used herein, a “synthetic composition” is a composition which is artificially prepared and is containing at least one compound that is produced ex vivo chemically and/or biologically and/or physically, e.g. by is means of chemical reaction, enzymatic reaction or by a fractionation process. An example of such a fractionation process is a process wherein bovine milk is separated into different fractions like a fat and protein fraction. For the avoidance of doubt, a synthetic composition is not made in vivo by man or animal.

Without wishing to be bound by any particular theory, the methods of the present invention enhance muscle performance and/or reduce chronic fatigue by improving mitochondrial function via administration of milk-derived EV (mEV) together with galactose to the subject in need thereof. Preferably the mEV are bovine mEV, more preferably bovine mEV sourced from a whey-containing bovine milk fraction.

As used herein, the term “galactose” refers to the monosaccharide D-galactose as such. The term does not refer to compounds containing a (covalently) bonded galactose moiety, such as proteins glycosylated with galactose.

Likewise, the term “glucose” refers to the monosaccharide D-glucose. The present inventors have surprisingly found that mEV with galactose significantly enhance both maximal respiratory capacity and spare respiratory capacity as compared to mEV without galactose or mEV with glucose, as shown in the examples. mEV with galactose can thus be administered to a subject to improve mitochondrial function. The improved mitochondrial function results in improved muscle performance, and/or reduced chronic fatigue. Treating mitochondrial dysfunction may thus be an effective way to alleviate fatigue following a viral infection and/or to treat fatigue due to sarcopenia.

The mEV preferably is mammalian mEV such as derived from bovine milk, sheep milk, goat milk, horse milk, camel milk or cow's milk; even more preferably derived from bovine milk, most preferably derived from cow's milk.

The composition of the invention may be considered as an mEV product additionally comprising galactose. The term “mEV-enriched product” as used herein, unless otherwise specified, refers to a product in which mEVs is have been substantially separated from other milk components such as lipids, cells, and debris, and are concentrated in an amount higher than that found in bovine milk. The mEV are small, extracellular vesicles and account for a minor percentage of milk's total solids content. In specific embodiments, the mEV-enriched product is provided in a liquid form or a powdered form and also contains co-isolated milk solids.

“Bovine” is referring to an animal of the cattle group and includes the antelopes, sheep, goats, cattle, buffalo, and bison, bovine is preferably referring to the domestic cattle group including sheep, goats, cattle, and buffalo.

The term “intact extracellular vesicle” as used herein refers to extracellular vesicles (EV) in which the vesicle membrane is not ruptured and/or otherwise degraded and as such a vesicle size may be determined using methods described elsewhere herein. The endogenous cargo, i.e., the bioactive agents, therapeutics (e.g. miRNA), and/or other biomolecules which are inherently present in a milk-derived extracellular vesicle (mEV), are retained in intact EV in active form.

In a particularly preferred embodiment, the mEV is an exosome. Extracellular vesicles may be ruptured during isolation and/or enrichment thereof. Accordingly, in one embodiment the mEV in the synthetic composition of the invention are comprising intact bovine mEV, preferably wherein the bovine mEV are sourced from a whey-containing bovine milk fraction.

Transmission electron microscopy (TEM) may be used for purposes of assessing the presence of mEV in an mEV-enriched product. TEM is a technique which can be used for the direct visualization of nanosized structures, such as mEV. Uranyl acetate may be applied as a negative dye to study the impact of thermal treatments, such as pasteurization, evaporation, spray-drying, and freeze-drying, on the mEV structure of the mEV in the product. Briefly, the uranyl acetate acts as a negative dye, which stains the background and leaves the intact vesicular structures, such as intact extracellular vesicles, unstained and highly visible (as is shown in WO2022146743).

In one embodiment, the mEV-enriched product of the invention further comprising galactose, e.g. the product of claim 1, comprises at least 0.001 wt % mEV as determined relative to the dry weight of the product. In another specific embodiment, the mEV-enriched product comprises at least about 0.001 wt %, 0.01 wt %, 1 wt %, 5 wt %, 10 wt %, 15 wt %, 20 wt %, 25 wt %, 30 wt %, 35 wt %, 40 wt %, 45 wt %, or 50 wt % mEV as determined relative to the dry weight of the product. In a further embodiment, the mEV-enriched product comprises at least about 10⁸ mEV per gram of the mEV-enriched product as measured by a nanotracking procedure. Briefly, nanoparticle tracking analysis (NTA) can be used to determine mEV diameter and concentration. The principle of NTA is based on the characteristic movement of nanosized particles in solution according to the Brownian motion. The trajectory of the particles in a defined volume is recorded by a camera that is used to capture the scatter light upon illumination of the particles with a laser. The Stokes-Einstein equation is used to determine the size of each tracked particle. In addition to particle size, this technique also allows determination of particle concentration.

In one embodiment, the mEV-enriched product of the invention further comprising galactose, e.g. the product of claim 1, comprises at least 0.1 wt % mEV as determined relative to the dry weight of the product.

In another specific embodiment, the mEV-enriched product of the invention comprises from about 10⁸ to about 10¹⁴ mEV per gram of the mEV-enriched product. In yet a more specific embodiment, the mEV-enriched product comprises from about 10⁹ to about 10¹³ mEV per gram of the mEV-enriched product. In another specific embodiment, the mEV enriched product contains at least about a three-fold increase in the number of mEV, as compared to a raw whey-containing bovine milk fraction. In another specific embodiment, the mEV-enriched product contains a 3-fold to 50-fold increase in the number of mEV, as compared to a raw whey-containing bovine milk fraction, for example cheese whey.

In yet another embodiment, the amount of galactose in the nutritional composition of the invention, i.e. the mEV enriched product, is at least 0.01 wt % galactose as determined relative to the dry weight of the product, preferably at least 0.1 wt %, more preferably at least 0.5 wt %. In an even more preferred embodiment, the amount of galactose in the nutritional composition of the invention is at least 1.0 wt % as determined relative to the dry weight of the product.

In a preferred embodiment, the wt % of galactose in the composition of the invention is 8 times higher than the wt % of mEV because only about 12% of the digested galactose arrives in the blood stream after passage through the digestive tract, hepatic vein and liver.

In another embodiment of the synthetic nutritional composition of the invention the amount of mEV is at least 0.001 wt % and the amount of galactose is at least 0.01 wt %, preferably the amount of mEV is at least 0.01 wt % and the amount of galactose is at least 0.01 wt %, more preferably the amount of mEV is at least 0.1 wt % and the amount of galactose is at least 1.0 wt %, most preferably wherein the amount of mEV is at least 1.0 wt % and the amount of galactose is at least 1.0 wt %, all wt % are determined relative to the dry weight of the composition.

In still another embodiment of the synthetic nutritional composition of the invention the diameter of greater than 90% of the bovine mEV is from about 10 nanometers to about 250 nanometers.

In yet another embodiment, at least 50 wt % of the mEV are intact, preferably wherein at least about 55, 60, 65, 70, 75, 80, 85, 90, or 95% of the mEV are intact. Preferably, the level of intact mEV is determined relative to level of mEV in the source material e.g. milk fractions, used to prepare the composition of the invention.

In another embodiment, the mEV-enriched product comprising galactose is for oral administration, preferably wherein the composition is a powder, a liquid or a bar.

The composition of the invention may further comprise one or more is selected from the group consisting of a protein fraction, a carbohydrate fraction, and a fat fraction. Optionally, the composition is comprising one or more nutrients selected from the group consisting of vitamins and minerals.

The mEV enriched product may be the sole source of protein in the nutritional composition of the invention. Nevertheless, additional protein sources can be included in the nutritional composition i.e. protein fraction. In one embodiment, the protein fraction comprises whole egg powder, egg yolk powder, egg white powder, whey protein, whey protein concentrates, whey protein isolates, whey protein hydrolysates, acid caseins, casein protein isolates, sodium caseinates, calcium caseinates, potassium caseinates, casein hydrolysates, milk protein concentrates, milk protein isolates, milk protein hydrolysates, nonfat dry milk, condensed skim milk, whole cow's milk, partially or completely defatted milk, coconut milk, soy protein concentrates, soy protein isolates, soy protein hydrolysates, pea protein concentrates, pea protein isolates, pea protein hydrolysates, rice protein concentrate, rice protein isolate, rice protein hydrolysate, fava bean protein concentrate, fava bean protein isolate, fava bean protein hydrolysate, collagen proteins, collagen protein isolates, meat proteins, potato proteins, chickpea proteins, canola proteins, mung proteins, quinoa proteins, amaranth proteins, chia proteins, hemp proteins, flax seed proteins, earthworm proteins, insect proteins, one or more amino acids and/or metabolites thereof, or combinations of two or more thereof.

The one or a mixture of amino acids, which may be described as free amino acids, can be any amino acid known for use in nutritional products.

The amino acids may be naturally occurring or synthetic amino acids. In a specific embodiment, the one or more amino acids and/or metabolites thereof comprise one or more branched chain amino acids or metabolites thereof. Examples of branched chain amino acids include arginine, glutamine leucine, isoleucine, and valine. In another specific embodiment, the one or more branched chain amino acids or metabolites thereof comprise alpha-hydroxy-isocaproic acid (HICA, also known as leucic acid), keto isocaproate (KIC), beta-hydroxy-beta-methylbutyrate (HMB), and combinations of two or more thereof.

The nutritional composition may comprise a protein fraction in an amount from about 1 wt % to about 50 wt %, such as from about 1 wt % to about 30 wt % of the nutritional composition. More specifically, the protein may be present in an amount from about 1 wt % to about 25 wt % of the nutritional composition, including about 1 wt % to about 20 wt %, about 2 wt % to about 20 wt %, about 1 wt % to about 15 wt %, about 1 wt % to about 10 wt %, about 5 wt % to about 10 wt %, about 10 wt % to about 25 wt %, or about 10 wt % to about 20 wt % of the nutritional composition. Even more specifically, the protein comprises from about 1 wt % to about 5 wt % of the nutritional composition, or from about 20 wt % to about 30 wt % of the nutritional composition. Alternatively, in yet another embodiment, the nutritional product is a high protein product comprising a protein fraction in an amount from about 20 wt % to about 90 wt %, preferably from 30 wt % to 80 wt %, more preferably from 35 wt % to 75 wt %.

As used herein, the carbohydrate fraction is not referring to the galactose of the composition of the invention. The carbohydrate fraction may comprise one or more selected from the group consisting of maltodextrin, starch, dextrose, dextrins, lactose, galactooligosaccharides, fructooligosaccharides, human milk oligosaccharides (HMOs), and galactomannan. Examples of starches that may be used include hydrolyzed starch, modified starch, cornstarch, and hydrolyzed cornstarch.

The nutritional composition may comprise carbohydrate in an amount from about 5 wt % to about 75 wt % of the nutritional composition. More specifically, the carbohydrate may be present in an amount from about 5 wt % to about 70 wt % of the nutritional composition, including about 5 wt % to about 65 wt %, about 5 wt % to about 50 wt %, about 5 wt % to about 40 wt %, about 5 wt % to about 30 wt %, about 5 wt % to about 25 wt %, about 10 wt % to about 65 wt %, about 20 wt % to about 65 wt %, about 30 wt % to about 65 wt %, about 40 wt % to about 65 wt %, about 40 wt % to about 70 wt %, or about 15 wt % to about 25 wt %, of the nutritional composition.

The fat fraction may comprise milk fat, cream, anhydrous milk fat, algal oil, canola oil, flaxseed oil, borage oil, safflower oil, high oleic safflower oil, high gamma-linolenic acid (GLA) safflower oil, corn oil, soy oil, sunflower oil, high oleic sunflower oil, cottonseed oil, coconut oil, fractionated coconut oil, medium chain triglycerides (MCT) oil, palm oil, palm kernel oil, palm olein, long chain polyunsaturated fatty acids, or combinations of two or more thereof.

The nutritional composition may comprise fat in an amount of from about 0.5 wt % to about 30 wt % of the nutritional composition. More specifically, the fat may be present in an amount from about 0.5 wt % to about 10 wt %, or from about 1 wt % to about 30 wt % of the nutritional composition, including about 1 wt % to about 20 wt %, about 1 wt % to about 15 wt %, about 1 wt % to about 10 wt %, about 1 wt % to about 5 wt %, about 3 wt % to about 30 wt %, about 5 wt % to about 30 wt %, about 5 wt % to about 25 wt %, about 5 wt % to about 20 wt %, about 5 wt % to about 10 wt %, or about 10 wt % to about 20 wt % of the nutritional composition.

In one embodiment, the nutritional composition is a liquid nutritional composition and comprises from about 1 to about 15 wt % of protein, from about 0.5 to about 10 wt % fat, and from about 5 to about 30 wt % carbohydrate, based on the weight of the nutritional composition.

In another embodiment, the nutritional composition is a powder nutritional composition and comprises from about 10 to about 30 wt % of protein, from about 5 to about 15 wt % fat, and from about 30 wt % to about 65 wt % carbohydrate, based on the weight of the nutritional composition.

In a specific embodiment, the nutritional composition comprises at least one protein comprising milk protein concentrate and/or soy protein isolate, at least one fat comprising milk fat, canola oil, corn oil, coconut oil and/or marine oil, and at least one carbohydrate comprising maltodextrin, sucrose, lactose, galactooligosaccharides and/or fructooligosaccharides.

The nutritional composition may also comprise one or more components to modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), colorants, flavorants, thickening agents, stabilizers, and so forth.

In specific embodiments, the nutritional composition has a neutral pH, i.e., a pH of from about 6 to 8 or, more specifically, from about 6 to 7.5. In more specific embodiments, the nutritional composition has a pH of from about 6.5 to 7.2 or, more specifically, from about 6.8 to 7.1. The nutritional composition may be formed using any techniques known in the art. In one embodiment, the nutritional composition may be formed by (a) preparing an aqueous solution comprising protein and carbohydrate; (b) preparing an oil blend comprising fat and oil-soluble components; and (c) mixing together the aqueous solution and the oil blend to form an emulsified liquid nutritional composition. The intact mEV may be added at any time as desired in the process, for example, to the aqueous solution or to the emulsified blend. The intact mEV may be dry blended in powder form with one or more dry ingredients, for example, for combined addition to a liquid composition or if a powdered nutritional product is desirable.

In a specific embodiment, the nutritional composition is administered in the form of a powder. In another specific embodiment, the nutritional composition is administered in the form of a liquid. The nutritional composition can be administered to the subject in either form.

When the nutritional composition is a powder, for example, a serving size is from about 40 g to about 60 g, such as 45 g, or 48.6 g, or 50 g, to be administered as a powder or to be reconstituted in from about 1 ml to is about 500 ml of liquid.

When the nutritional composition is in the form of a liquid, for example, reconstituted from a powder or manufactured as a ready-to-drink product, a serving ranges from about 1 ml to about 500 ml, including from about 110 ml to about 500 ml, from about 110 ml to about 417 ml, from about 120 ml to about 500 ml, from about 120 ml to about 417 ml, from about 177 ml to about 417 ml, from about 207 ml to about 296 ml, from about 230 m to about 245 ml, from about 110 ml to about 237 ml, from about 120 ml to about 245 ml, from about 110 ml to about 150 ml, and from about 120 ml to about 150 ml. In specific embodiments, the serving is about 1 ml, or about 100 ml, or about 225 ml, or about 237 ml, or about 500 ml.

In specific embodiments, the nutritional compositions comprising bovine mEV are administered to a subject once or multiple times daily or weekly.

In specific embodiments, the nutritional composition is administered to the subject from about 1 to about 6 times per day or per week, or from about 1 to about 5 times per day or per week, or from about 1 to about 4 times per day or per week, or from about 1 to about 3 times per day or per week. In specific embodiments, the nutritional composition is administered once or twice daily for a period of at least one week, at least two weeks, at least three weeks, or at least four weeks.

The concentration and relative amounts of the protein fraction, carbohydrate fraction, and fat fraction in the nutritional compositions can vary considerably depending upon, for example, the specific dietary needs of the intended user. In a specific embodiment, the nutritional composition comprises a source of protein in an amount of about 2 wt % to about 20 wt %, a source of carbohydrate in an amount of about 5 wt % to about 30 wt %, and a source of fat in an amount of about 0.5 wt % to about 10 wt %, based on the weight of the nutritional composition, and, more specifically, such composition is in liquid form. In another specific embodiment, the nutritional composition comprises a source of protein in an amount of about 10 wt % to about 25 wt %, a source of carbohydrate in is an amount of about 40 wt % to about 70 wt %, and a source of fat in an amount of about 5 wt % to about 20 wt %, based on the weight of the nutritional composition, and, more specifically, such composition is in powder form.

In one aspect the invention relates the synthetic nutritional composition of the invention for use in enhancing muscle performance in a subject and/or for use in increasing muscle growth in a subject, preferably in a subject in need of improved physical performance.

In yet another aspect, the invention relates to the use of the composition of the invention for enhancing muscle performance in a subject and/or the use of increasing muscle growth in a subject, preferably in a subject in need of improved physical performance.

In still another aspect, the invention relates to the use of the composition of the invention in the manufacture of a medicament for enhancing muscle performance and/or increasing muscle growth.

In one embodiment the daily dose of the composition for use of the invention is between 0.01 to 30 g mEV per day, preferably between 0.1 and 20 g mEV per day. More preferably, the daily dose is between 0.01 and 30 g mEV per day and between 0.01 and 30 g galactose per day, even more preferably the daily dose is between 0.1 and 20 g mEV per day and between 1.0 and 25 g galactose per day.

It must be noted that, as used in the specification and the appended claims, the singular form “a”, “an,” and “the” comprise plural referents unless the context clearly indicates otherwise. For example, reference to a component in the singular is intended to comprise a plurality of components.

It will be understood that within this disclosure, any reference to a weight, weight ratio, and the like pertains to the dry matter, in particular the dry matter of the composition, unless defined otherwise.

Unless defined otherwise, all technical and scientific terms used herein generally have the same meaning as commonly understood by one of ordinary skill in the art to which this invention belongs.

To the extent that the term “includes” or “including” is used in the description or the claims, it is intended to be inclusive of additional elements or steps, in a manner similar to the term “comprising” as that term is interpreted when employed as a transitional word in a claim.

As used herein, the term “comprising”, which is synonymous with “including” or “containing”, is open-ended, and does not exclude additional, unrecited element(s), ingredient(s) or method step(s), whereas the term “consisting of” is a closed term, which excludes any additional element, step, or ingredient which is not explicitly recited.

Furthermore, to the extent that the term “or” is employed (e.g., A or B), it is intended to mean “A or B or both.” When the “only A or B but not both” is intended, then the term “only A or B but not both” is employed. Thus, use of the term “or” herein is the inclusive, and not the exclusive use. When the term “and” as well as “or” are used together, as in “A and/or B” this indicates A or B as well as A and B.

Throughout this application, where publications are referenced, the disclosures of these publications in their entireties are hereby incorporated by reference into this application to more fully describe the state of the art to which this invention pertains.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical is interactions among the constituents of a mixture once mixed; the first definition of an acronym or other abbreviation applies to all subsequent uses herein of the same abbreviation and applies, mutatis mutandis, to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described herein, as specific components and/or conditions may, of course, vary. Furthermore, the terminology used herein is used only for the purpose of describing particular embodiments of the present invention and is not intended to be limiting in any way.

The invention is hereinafter illustrated with reference to the following, non-limiting, examples.

### Examples

### Example 1 mEV Isolation

Commercial pasteurized skimmed cow's milk was purchased at the local supermarket and stored at 4° C. until mEV isolation (max one day storage). Milk samples were centrifuged at 70,000×g for 60 min at 4° C., to remove milk fat globules, residual milk fat globules, casein proteins, and other debris (Polypropylene tubes, Thermo Fisher AH-629 rotor—Max acceleration, no break). The clear supernatant was subsequently filtered through Whatman papers, first #1 and thereafter #50, and finally through 0.45 and 0.2 μm syringe filters. mEVs were isolated from the filtered supernatant by ultracentrifugation at 110,000×g for 90 min at 4° C. without breaks. mEVs, located on top of a firm casein pellet, were washed and taken up in PBS or in the appropriate buffer for RNA or protein analysis. For each isolation roughly 40 mL of milk was processed, resulting is in 1 mL of EVs (protein—particle ratio 5-15 fg particle⁻¹). After isolation, EVs were aliquoted and stored at 4° C. for up to 6 weeks. The amount of protein for each isolation was measured with a Micro-BCA kit (Thermo Scientific, Pierce, Rockford, USA). This isolation procedure was as described by B. C. H. Pieters, et al (Bovine Milk-Derived Extracellular Vesicles Inhibit Catabolic and Inflammatory Processes in Cartilage from Osteoarthritis Patients; B. C. H. Pieters, et al, Mol. Nutr. Food Res. 2022, 2100764; DOI: 10.1002/mnfr.202100764).

### Samples Used in the Seahorse Experiment

The samples used in the Seahorse experiment are listed in Table 1. Glucose i.e. D-glucose, and galactose i.e. D-galactose, were obtained from Sigma-Aldrich.

TABLE 1
composition of the samples tested

| Sample | Compound |
|---|---|
| 1 | mEV 50 μg/ml |
| 2 | mEV 100 μg/ml |
| 3 | Glucose 1.4 mM |
| 4 | Glucose 2.75 mM |
| 5 | Glucose 5.5 mM |
| 6 | Galactose 1.4 mM |
| 7 | Galactose 2.75 mM |
| 8 | Galactose 5.5 mM |
| 9 | mEV 50 μg/ml + Glucose 1.4 mM |
| 10 | mEV 50 μg/ml + Glucose 2.75 mM |
| 11 | mEV 50 μg/ml + Glucose 5.5 mM |
| 12 | mEV 50 μg/ml + Galactose 1.4 mM |
| 13 | mEV 50 μg/ml + Galactose 2.75 mM |
| 14 | mEV 50 μg/ml + Galactose 5.5 mM |

Commonly identified microRNAs in mEVs.

### MicroRNA Present in

| bovine mEV | Expected function |
|---|---|
| Let7 | Protection against bacterial infection |
| miR-21 | Linked to regulation of TLR signaling |
| | Clearance of apoptotic cells |
| | Clearance of bacterial infection |
| miR-146 | Linked to regulation of TLR signaling |
| | Clearance of bacterial infection |
| miR-148 | Inhibition of demethylation Foxp3 |
| | Suppression of TGFb signaling via SMAD |
| | Regulation of DNMT1 and DNMT3, epigenetic |
| | homeostasis of DNA methylation |
| miR-155 | Anti-inflammatory effects |
| | Regulation of TLR signaling |
| | Induction of Tregs |
| miR-181 | Anti-inflammatory effects |
| | NFkB signaling |
| miR-223 | Linked to infection and inflammation |
| | Eosinophil function |

In one embodiment the mEV-comprising composition of the invention is comprising one or more of micro RNAs selected from the group consisting of Let7, miR-21, miR-146, miR-148, miR-155, miR-181, and miR-223; preferably it is comprising 2 or more micro RNAs selected from the group consisting of Let7, miR-21, miR-146, miR-148, miR-155, miR-181, and miR-223; more preferably three or more micro RNAs selected from the group consisting of Let7, miR-21, miR-146, miR-148, miR-155, miR-181, and miR-223.

Preferably the mEV as used in the different aspects and embodiments of the invention are pasteurized mEV, more preferably, the mEV are heat-treated at least at 72° C. for 15 s.

### Example 2. Enhanced Maximal Respiratory Capacity and Mitochondrial SRC in C2C12 Myoblasts Incubated with Intact Bovine Milk-Derived Extracellular Vesicles

The execution of the “Seahorse” experiment as used in this example is explained in more detail in Traba et al. (2016) J. Vis. Exp. (117), e54918 especially in FIG. 1 and text relating thereto, and in Rose S, et al. (2014) Oxidative Stress Induces Mitochondrial Dysfunction in a Subset of Autism Lymphoblastoid Cell Lines in a Well-Matched Case Control Cohort. PLoS ONE 9(1): e85436. doi:10.1371/journal.pone.0085436. Briefly, in the Seahorse essay, the Oxygen Consumption Rate (OCR) is measured before and after the addition of inhibitors to derive several parameters of mitochondrial respiration. Initially, baseline cellular OCR is measured, from which basal respiration can be derived by subtracting non-mitochondrial respiration. Next oligomycin, a complex V inhibitor, is added and the resulting OCR is used to derive ATP-linked respiration (by subtracting the oligomycin rate from baseline cellular OCR) and proton leak respiration (by subtracting non-mitochondrial respiration from the oligomycin rate). Next carbonyl cyanide-p-trifluoromethoxyphenyl-hydrazon (FCCP), a protonophore, is added to collapse the inner membrane gradient, allowing the ETC to function at its maximal rate, and maximal respiratory capacity is derived by subtracting nonmitochondrial respiration from the FCCP rate. Lastly, antimycin A and rotenone, inhibitors of complex III and I, are added to shut down ETC function, revealing the non-mitochondrial respiration. Mitochondrial reserve capacity is calculated by subtracting basal respiration from maximal respiratory capacity.

C2C12 cells were routinely cultured in High Glucose Dulbecco's Modified Eagle's Medium (DMEM) supplemented with 10% (v/v) fetal calf serum (FCS), 2 mM glutamine, 25 mM HEPES pH 7.2 and 1% (v/v/v) pen/strep/amphotericin B at 5% CO₂ and 37 degrees Celsius. For Seahorse experiments, C2C12 myoblasts (passage 10-20, ATCC) were plated in the morning in XFe96 Seahorse culture plates at a density of 30.000 cells/well. Cells were kept in the flow cabinet for 1 h before being transferred to the incubator. After approximately 6-8 h cells, culture medium was replaced with C2C12 differentiation medium (DMEM with 2% horse serum). C2C12 cells were allowed to differentiate for 5 to 7 days.

On the day before the Seahorse run, myotubes were exposed to different nutrients and/or Extracellular Vesicles (mEVs) for 24 hours.

mEVs were provided by FrieslandCampina and isolated as described in Example 1. On the day of delivery, the tube containing liquid with EVs was stored immediately in the fridge at 4 degrees Centigrade and mEVs were used within two weeks after delivery.

Exposure medium was prepared in C2C12 differentiation medium. Galactose and glucose were supplemented to differentiation medium from 1M stocks in H₂O. For mEV exposure 150 ul or 300 ul of a mEV stock solution (1000 μg/ml in 1×PBS) was diluted in 3 ml differentiation medium and exposed to C2C12 myotubes in plates. As controls, 1×PBS without mEV was diluted in culture medium at 150 ul and 300 ul in 3 ml culture medium.

Seahorse experiments were performed using the XFe96 extracellular flux analyzer (Agilent) at 37 degrees Celsius. Cells were assayed in Seahorse DMEM without FBS and without PS. The Seahorse assay medium consisted of DMEM with HEPES (Agilent) with 10 mM glucose (Agilent), 1 mM pyruvate (Agilent) and 2 mM glutamine (Agilent) at ph 7.4. A mito-stress test was performed according to manufacturer's instructions. In detail, following calibration and equilibration, plates were analyzed in measure/mix cycles of 3 minutes/2 minutes, respectively, in 180 ul Seahorse assay medium. After baseline measurements, an injection strategy of 20 ul of 15 uM oligomycin, 22 ul FCCP 15 uM and 25 ul antimycin (25 uM)/rotenone (12.5 uM) was performed with after each is injection three measure/mix cycles. All chemicals for the mito-stress test were obtained from Sigma.

In the final injection the nuclear fluorescent stain, Hoechst (40 uM), was also injected, to allow for cell number quantification. For this, all wells of a plate were automatically imaged using a Cytation 1 (Biotek, Agilent), immediately after the Seahorse run was finished. Images were analyzed using in-house generated scripts in ImageJ and R for processing data. All Seahorse data were normalized to cell number using the nuclear stain normalization method.

Mitochondrial function was analyzed by measuring the OCR in the C2C12 myotubes using the SeaHorse XFe24 flux analyzer with XF Cell Mito Stress kit in accordance with manufacturer instructions. A measurement of basal respiration was taken and recorded. lonophore carbonylcyanide p-trifluoromethoxyphenylhydrazone (FCCP) (1.5 mM) was then injected to measure maximal respiratory capacity (MAX), which was also recorded. FCCP mimics a physiological “energy demand” by stimulating the respiratory chain to operate at maximum capacity, so the OCR observed after the addition of the ionophore corresponds to the maximal respiration level. The FCCP-stimulated OCR can then be used to calculate Spare Respiratory Capacity (SRC), which, as described above, is defined as the difference between maximal respiration and basal respiration.

This example demonstrates that an mEV-enriched product containing mEV enhances maximal respiratory capacity and mitochondrial SRC in C2C12 myoblasts. Mitochondrial function was analyzed by measuring the oxygen consumption rate (OCR) in differentiated C2C12 myotubes incubated with either powdered bovine milk-derived mEV resuspended in phosphate buffer saline (PBS) or with PBS alone using the Sea Horse flux analyzer in accordance with manufacturer instructions.

Maximal respiratory capacity is increased by mEV, as shown by an increase in basal OCR (also referred to as basal respiration) in the is Seahorse experiment, as illustrated in FIG. 1 which shows the basal respiration (OCR) of sample 1 (second bar from the right) and sample 2 (right bar) as compared to control (C, left bar) and vehicle control (PBS; 5 and 10% of total volume; second and third bar from the left).

Glucose and galactose each increase basal respiration as shown in FIG. 2 (galactose sample 6, 7, and 8 shown in 1^{st}, 3^{rd}, and 5^{th} bar, respectively, glucose sample 3, 4, and 5 shown in 7^{th}, 9^{th}, and 11^{th} bar, respectively. A higher concentration of galactose or glucose increases basal respiration. Surprisingly, addition of mEV to galactose significantly increases basal respiration, while the addition of mEV to glucose has no effect as illustrated in FIG. 2, 2^{nd}, 4^{th}, and 6^{th} bar from the left as compared to 8^{th}, 10^{th}, and 12^{th} bar from the left, representing samples 12, 13, 14, 9, 10, and 11, respectively. In particular, it is shown that sample 12, as shown in FIG. 2, 2^{nd} bar from the left, representing a low amount of mEV (50 ug/mlof mEV) combined with a low amount of galactose (1.4 mM) has about the same basal respiration as all other combinations of mEV with either glucose or galactose.

These results thus indicate that the intact bovine mEVcan improve mitochondrial function and therefore enhance muscle performance, which is particularly relevant for the prevention, treatment or recovery of conditions that are hallmarked by a reduction in SRC, such as sarcopenia and chronic or acute cardiac damage. As indicated above, treating mitochondrial dysfunction is also an effective way to alleviate chronic fatigue following a viral infection. The indication that the intact bovine mEV can improve mitochondrial function is particularly relevant for improving chronic fatigue associated with the recovery from a viral infection.

1: A synthetic nutritional composition comprising galactose and milk-derived extracellular vesicles (mEV).

2: The synthetic nutritional composition of claim 1 wherein the mEV are mammalian mEV, preferably bovine mEV, more preferably cow's mEV.

3: The synthetic nutritional composition of claim 1 wherein the mEV are comprising intact bovine mEV, preferably wherein the bovine mEV are sourced from a whey-containing bovine milk fraction.

4: The synthetic nutritional composition of claim 1 comprising at least 0.001 wt % mEV as determined relative to the dry weight of the product.

5: The synthetic nutritional composition of claim 1 comprising at least 0.1 wt % mEV as determined relative to the dry weight of the product.

6: The synthetic nutritional composition of claim 1 wherein the amount of galactose is at least 0.01 wt % as determined relative to the dry weight of the product.

7: The synthetic nutritional composition of claim 1 wherein the amount of galactose is at least 1.0 wt % as determined relative to the dry weight of the product.

8: The synthetic nutritional composition of claim 1 wherein the amount of mEV is at least 0.001 wt % and the amount of galactose is at least 0.01 wt %, preferably the amount of mEV is at least 0.01 wt % and the amount of galactose is at least 0.01 wt %, more preferably the amount of mEV is at least 0.1 wt % and the amount of galactose is at least 1.0 wt %, most preferably wherein the amount of mEV is at least 1.0 wt % and the amount of galactose is at least 1.0 wt %, all wt % are determined relative to the dry weight of the composition.

9: The synthetic nutritional composition of claim 1, wherein greater than 90% of the bovine mEV is from about 10 nanometers to about 250 nanometers in diameter.

10: The synthetic nutritional composition of claim 1, wherein the composition is comprising one or more micro RNAs selected from the group consisting of Let7, miR-21, miR-146, miR-148, miR-155, miR-181, and miR-223.

11: The synthetic nutritional composition of claim 1, wherein the composition is for oral administration, preferably wherein the composition is a powder, a liquid or a bar.

12: The synthetic nutritional composition of claim 1, wherein the composition is further comprising one or more selected from the group consisting of a protein fraction, a carbohydrate fraction, and a fat fraction; and optionally wherein the composition is comprising one or more nutrients selected from the group consisting of vitamins and minerals.

13: The synthetic nutritional composition of claim 1 for use in enhancing muscle performance in a subject and/or for use in increasing muscle growth in a subject.

14: The synthetic nutritional composition for use of claim 13 wherein the subject is in need of improved physical performance.

15: The synthetic nutritional composition for use of claim 12 wherein the daily dose of mEV is between 0.01 to 30 g mEV per day.
